(19) United States
(12) Patent Application Publication
(10) Pub. No.: US 2023/0075566 A1
(43) Pub. Date: Mar. 9, 2023

(54) INVENTORY MANAGEMENT SYSTEM

(71) Applicant: Gary L. Sharpe, Naples, FL (US)

(72) Inventors: Kurt Wolf, Dublin, OH (US); Stephen Stock, Hilliard, OH (US)

(21) Appl. No.: 17/899,153

(22) Filed: Aug. 30, 2022

Related U.S. Application Data
(60) Provisional application No. 63/238,573, filed on Aug. 30, 2021.

(51) Int. Cl. G16H 40/20; G06Q 10/08

(52) U.S. Cl. CPC G16H 40/20; G06Q 10/087

(57) ABSTRACT

Web-based systems and methods for monitoring various types of inventory, including inventory containing RFID tags or barcodes. Pre-tagged items are input into the system automatically upon being detected. The location of inventory carts, kits, and trays containing one or more items may be identified, monitored, and adjusted during use, deployment, and relocation. Inventory may be input using inventory bins with user-configurable par levels for ensuring sufficient stock levels, and the content of bins is monitored to address missing or misplaced items. Notifications about expirations and recalled items are provided to users.

### CROSS-REFERENCE TO RELATED APPLICATIONS

This application claims priority to U.S. Provisional Application No. 63/238,573 filed Aug. 30, 2021, the content of which is hereby incorporated by reference as if fully recited herein.

### TECHNICAL FIELD

Exemplary embodiments of the present invention relate generally to inventory management systems and methods, and more specifically, to web-based systems and associated methods for tracking and managing a wide range of items in a healthcare setting.

### BACKGROUND AND SUMMARY OF THE INVENTION

Tracking and managing inventory in any facility with various staff, services, and job responsibilities presents challenges and difficulties. In a healthcare setting, such as a hospital, clinic, surgical center, or other healthcare facility, the ability to inventory, track, and monitor the status of various items is particularly important. Not only must staff have any needed items on hand for planned medical procedures such as surgeries, but staff must also have access to needed items in times of emergencies and unplanned circumstances.

Many healthcare facilities such as hospitals utilize crash carts (also called code carts) to transport and dispense medication and equipment needed for certain types of medical and surgical emergencies. Crash carts may house medication and equipment for life support protocols. For example, a crash cart may contain drugs for rapid intubation, venous access, drugs for the treatment of common problems (e.g., adenosine, dextrose, epinephrine, naloxone), drugs for advanced cardiac life support. A crash cart may contain equipment such as but not limited to scissors, syringes, monitors, suction devices, bag valve masks. The contents of a crash cart may include items intended for use on children, and/or use on adults. Crash carts typically contain multiple drawers or shelves on which items can be placed.

Items intended to be used together for a procedure, or otherwise associated with a common application may be packaged together in the form of a tray or kit. Trays and kits may be sealed shut and/or locked once they are properly stocked/restocked. One example is a kit containing equipment for cardiac life support for an adult. Another example is a kit containing airway equipment for a child. As yet another example, a tray may contain different sizes of oxygen face masks. Kits and trays may contain only medication, only equipment, or contain both mediation and equipment. Healthcare facilities often decide how they wish to organize their crash carts and contents of the carts. It is common for certain drawers of a crash cart or even entire crash carts to be dedicated to certain types of patients or certain uses. For example, drawers for pediatric patients, or crash carts that are only for cardiac arrest related procedures. Crash carts may be restocked or inventoried in one or more locations (such as a pharmacy or central supply) and then dispatched to their designated area. Depending on the facility, during the course of a month or other period of time a particular crash cart may be repeatedly restocked and dispatched for use at a variety of locations. Accordingly, it can be difficult to locate any particular crash cart, kit, or tray within a facility, especially once it has been restocked and dispatched to a location for potential use.

Regardless of how items are ultimately organized, it is crucial that their presence, location, and important information about each item be monitored, and that such information can be accessed in real-time. In an emergency or other acute setting, the inability to locate a needed medication or piece of equipment necessary for a procedure can cause disruption, delay, or other severe consequences that may jeopardize a patient's health and the overall quality of services of a healthcare provider. The expiration date of medication must also be tracked to ensure that a patient does not receive an expired drug, or that the discovery of an expired drug in an emergency or acute setting does not cause delay or disruption while an unexpired substitute is located. Many health care facilities need to not only know expiration status (expired, or unexpired) but to have the ability to track upcoming expirations, or even set their own internal protocols for how far in advance of an expiration date an item should be removed from inventory. Of course, the amount of advance notice may change depending on the routine turnover of the particular item, which may depend on a variety of factors such as the type of item, the intended use of the item, whether it is part of a kit, which crash cart the item is in, and what location the crash cart is in. As an example, a vial of adenosine in a crash cart in a hospital's main trauma bay may experience greater turnover than a vial of adenosine that is kept in an overflow operating room used only on rare occasions. Recalled items must also be identified so that they can be quickly removed from circulation and use. In a scenario where there are multiple items subject to recall in a facility, it can be difficult to locate where each item is in order to remove it from circulation.

Item shortages can also be difficult to manage in any healthcare setting. Whether a shortage involves medicine, medical supplies, or other necessary equipment, a shortage can cause delay and disruption even when there is an approved substitute since it can be difficult to communicate to all necessary persons what an approved substitute is. It can also be difficult to keep track of all substitute items during the pendency of the shortage. Further complicating matters is the fact that in many situations it may be the case that the extent of the shortage is of a degree that only impacts some users, as the original item is present in some quantity. Communicating shortages, substitutes, and information pertaining to substitutes (such as the reason why a substitution may have to occur) can also be difficult when there are many persons involved, some of whom may work in different departments, or work during different shifts.

Different means are used to track items in a facility, including manual tracking, bar-code, and RFID technology. Manual tracking means that each item is visually reviewed by a person and any necessary information about each item is manually written down by a user. For example, the contents of crash carts, kits, trays, inventory bins, or any other receptacles holding medicine or medical supplies are kept in writing and updated by hand.

Barcode and RFID technology allow users to use a barcode or RFID reading technology to perform inventory checks and obtain information about a particular item. Of course, this only works to the extent that bar codes or RFID tags have been placed on items. In reality, many healthcare facilities need to track and monitor a wide range of items that do not all contain a barcode, or do not all have an associated RFID tag. For example, while items coming from a pharmacy department (e.g., drugs) may have barcodes, items coming from central supply (e.g., surgical tools) may not. In other circumstances many items in a pharmacy may be tagged with RFID tags, but non-pharmacy items may not. Accordingly, many facilities have a mix of items that are barcoded and/or RFID-tagged, or lacking either. Available inventory systems that are solely based on barcode technology or RFID technology fail to provide solutions that can monitor the wide array of items that most healthcare facilities work with.

The inventive concepts described herein address these and many other unmet needs. Described herein are exemplary web-based systems and associated methods for tracking and managing inventory, which can incorporate items that have barcodes, items that bear RFID tags, and items that have neither. The systems and methods described herein provide great flexibility in configuring rules for crash carts, drawers, kits, and trays, as well as flexibility in establishing rules for item shortages and substitutions. Individual items can be located in real-time, as well as larger assets such as crash carts, kits, and trays. Users can input items into inventory using inventory bins, and set par levels to maintain sufficient stock and anticipate any near-term needs as items are subsequently placed into kits, trays, or otherwise used. Systems and methods described herein support facilities and enterprises where many users need access to information, yet different groups of users have different roles and permissions. Certain activities, such as inputting items into the system, or restocking kits, may require approvals from certain authorized users before they can go into effect.

The system provides users with great flexibility to configure various attributes. Users can configure permitted uses and features of crash carts, drawers, kits, and furthermore, users can associate each asset to a permitted location. Users can also configure informational displays, generate custom reports, and otherwise configure the system in a variety of ways to meet their needs and responsibilities. Systems and methods provide novel means for locating and relocating both individual items and larger assets. Users can access the system through a web application, allowing for mobile access to the system over the internet. The application communicates with a central server associated with a database that stores all information related to items, as well as user roles, permissions, and configurations. RFID readers may communicate directly with the application server or with the central server. Barcode scanners may be operatively connected with a user device such as a desktop computer. The system may be configured to only accept certain types of data input via a barcode scanner, in order to reduce chances of user error when inputting information manually.

### BRIEF DESCRIPTION OF THE DRAWINGS

Novel features and advantages of the present invention, in addition to those mentioned above, will become apparent to those skilled in the art from a reading of the following detailed description in conjunction with the accompanying drawings wherein identical reference characters refer to identical parts and in which:

FIG. 1 is an example dashboard screen according to an exemplary embodiment;

FIG. 2 is an example Items List screen according to an exemplary embodiment;

FIG. 3 is the example screen of FIG. 2 showing a Configuration window;

FIG. 4 is an example Create Recall screen according to an exemplary embodiment;

FIG. 5 is an example Recalls screen according to an exemplary embodiment;

FIG. 6 is an example Items screen according to an exemplary embodiment;

FIG. 7 is an example Create Shortage screen according to an exemplary embodiment;

FIG. 8 is the example Create Shortage screen of FIG. 7;

FIG. 9 is an example Create Items screen according to an exemplary embodiment;

FIG. 10 is an example Bins screen according to an exemplary embodiment;

FIG. 11 is the example Bins screen according to FIG. 10;

FIG. 12 is an example Edit Bin screen according to an exemplary embodiment;

FIG. 13 is an example Bins screen according to an exemplary embodiment;

FIG. 14 is an exemplary Cycle Count results screen according to an exemplary embodiment;

FIG. 15 is the results screen according to FIG. 14 further displaying a Remove Selected Items popup window according to an exemplary embodiment;

FIG. 16 is an example Kits screen according to an exemplary embodiment;

FIG. 17 is an example Restock screen according to an exemplary embodiment;

FIG. 18 is an example Restocking Adult Tray screen according to an exemplary embodiment;

FIG. 19 is an example Restocking Adult Tray screen according to an exemplary embodiment;

FIG. 20 is an example Kit Approval screen according to an exemplary embodiment;

FIG. 21 is an example Crash Cart screen according to an exemplary embodiment;

FIG. 22 is an example Cart Types screen according to an exemplary embodiment;

FIG. 23 is an example Edit Cart Type screen according to an exemplary embodiment, further displaying an Edit Drawer window;

FIG. 24 is a Relocate screen according to an exemplary embodiment;

FIG. 25 is an Operating Room screen according to an exemplary embodiment;

FIG. 26 is a Create Items screen according to an exemplary embodiment;

FIG. 27 is a Reports screen according to an exemplary embodiment;

FIG. 28 is an exemplary Schedule Report screen according to an exemplary embodiment;

FIG. 29 is a Scheduled Reports screen according to an exemplary embodiment;

FIG. 30 is an exemplary mobile Dashboard screen according to an exemplary embodiment;

FIG. 31 is an exemplary mobile Kits screen according to an exemplary embodiment;

FIG. 32 is a mobile Cart Types screen according to an exemplary embodiment;

FIG. 33 is a mobile Restock screen according to an exemplary embodiment;

FIG. 34 is an exemplary system architecture screen according to an exemplary embodiment.

### DETAILED DESCRIPTION OF THE INVENTION

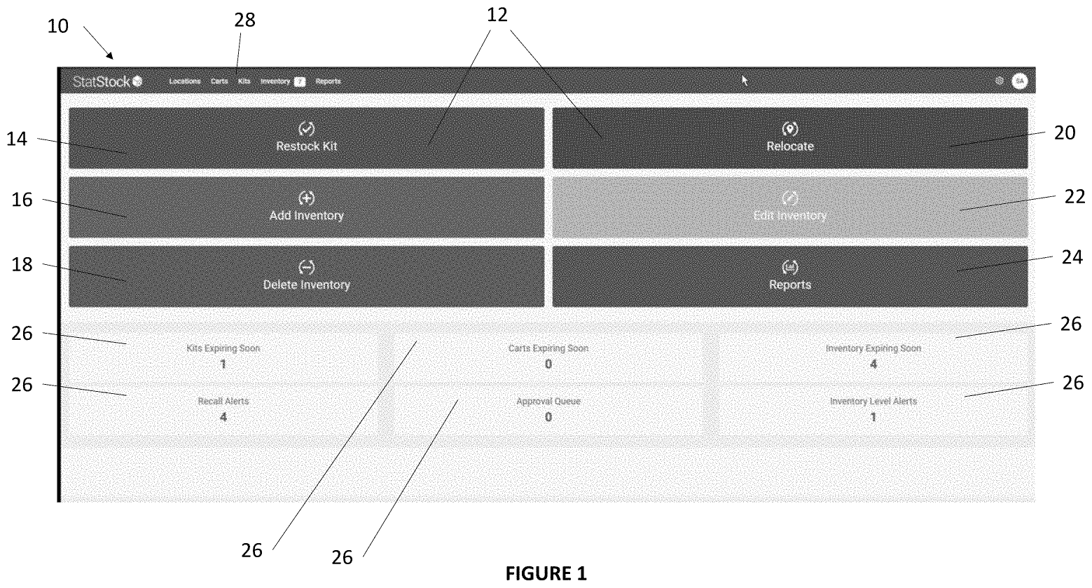

FIGS. 1 through 33 depict exemplary web application pages according to an embodiment of an inventory management system. These pages may be generated via an application server or web server, and accessed using a user device. The web application allows a plurality of users to view a wide variety of information and perform activities including, but not limited to, inputting information, generating reports, and running queries. All information collected by the system from user devices, peripherals such as barcode readers, or RFID readers, or generated by the system, may be stored in a database. Exemplary user devices include but are not limited to desktop computers, laptops, tablet computers, smart phones, and any other form of portable electronic device that is able to communicate with a web server over a network.

Referring to FIG. 1, an exemplary Dashboard page 10 contains a plurality of activity tabs 12, where the selection of each tab can initiate various activities. Included on the Dashboard page 10 are activity tabs 12 for restocking a kit 14, adding inventory 16, deleting inventory 18, relocating items 20, editing existing inventory 22, and viewing and generating reports 24. The Dashboard also includes informational tiles 26 that may display to a viewer highlights about current inventory. The informational tiles 26 may display information including, but not limited to, how many kits will have an expiration event within a predetermined amount of time, how many crash carts will have an expiration event within a predetermined amount of time, what inventory will have an expiration event within a predetermined amount of time, how many recall alerts are present, how many approvals are in the queue, and how many inventory level alerts are present. The Dashboard may provide a convenient way to access information and initiate the system to perform various operations. The Dashboard also provides a user with a high-level view of inventory status and an easy way to draw attention to any issues that need attention. For example, a user may view the Dashboard and determine how many work hours from a particular shift may need to be devoted to removing items from inventory that have an expiration event coming soon. In an exemplary embodiment a user can configure what type of information is displayed on the informational tiles. Furthermore, depending on a user's role and permissions in the system, different types of information may or may not be presented. The system can store user roles and permissions in a database and distinguish between different users based on their login information (e.g., user names and passwords). For example, if a particular user does not have permission to perform approvals, then the number of approvals in queue or the approvals requested by other users may not be displayed. In addition to the activity tabs 12 and informational tiles 26, a user may also select a variety of menu links 28 at the top of the page 10 to view information. Menu links may include, but not be limited to, Locations, Carts, Kits, Inventory, and Reports.

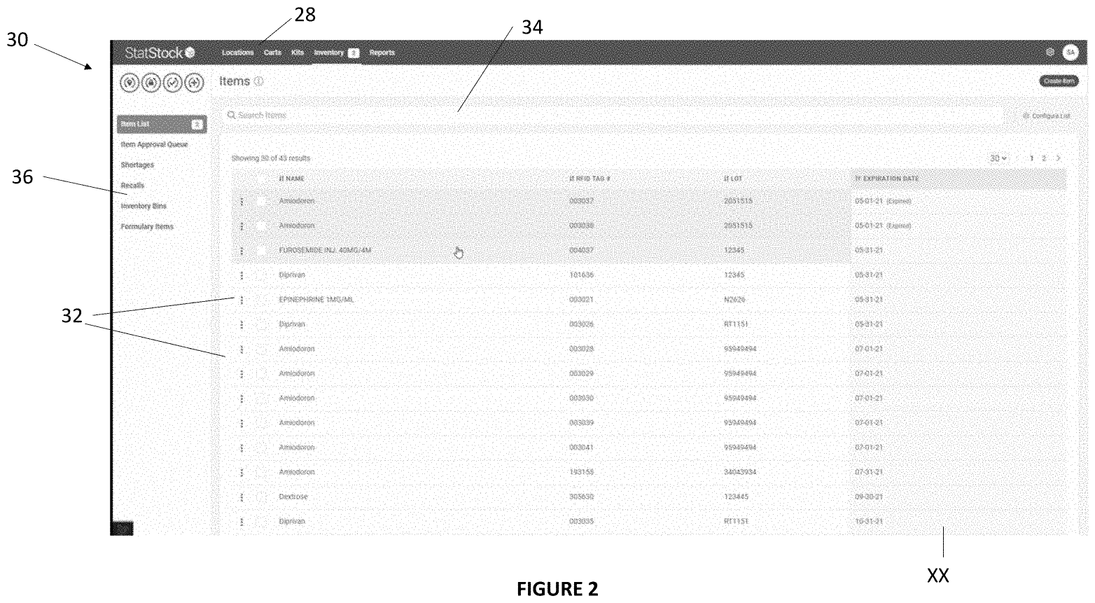

Referring to FIG. 2, an exemplary Items page 30 displays information about the various items that have been added to the system. The Inventory page 30 may be accessed through menu links 28. For each item in the system, the Inventory page may display information in an item listing 32 that includes, but is not limited to, the item's name, an RFID tag number (if the item has been tagged with an RFID tag), a lot number, and an expiration date. Depending on the item, certain fields may not apply and may be blank. For example, items that are not medications, such as supplies, may not have an expiration date. A search bar 34 at the top of the page allows a user to perform searches for items of interest, and a side bar 36 allows a user to access different sources of information and/or functions, such as Item List, Item Approval Queue, Shortages, Recalls, Inventory Bins, and Formulary Items.

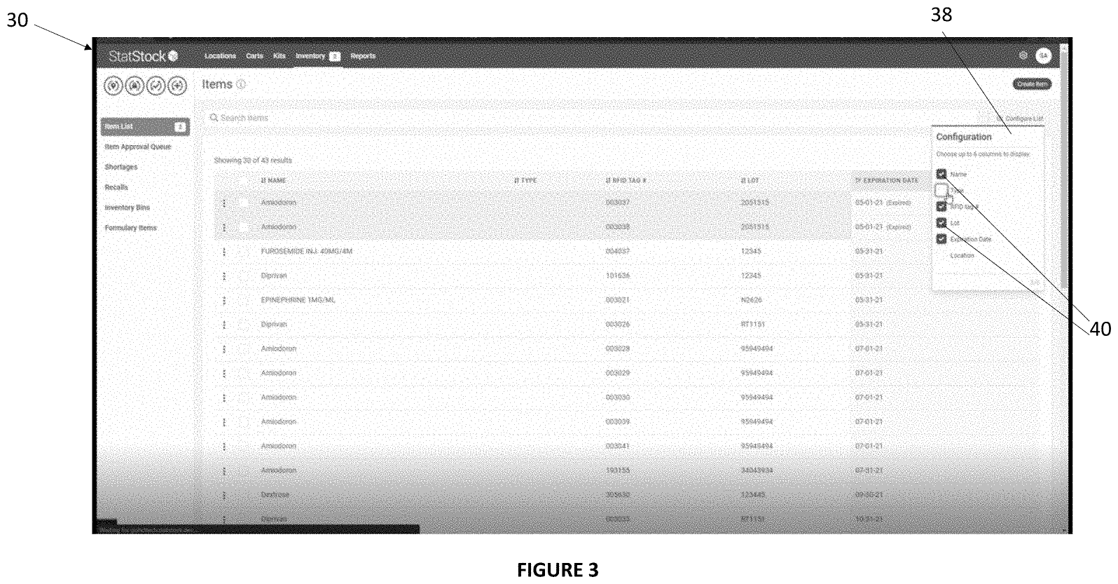

A user may select which types of information to display on the Items page. Referring to FIG. 3, a Configuration window 38 contains check boxes 40 that can be selected to display different columns of information. Configuration window 38 allows a user to select up to six columns of information for display, but in other embodiments different numbers of columns may be displayed. Configuration window 38 allows a user to display the following columns of information: Name, Type, RFID Tag #, Lot, Expiration Date, and Location. “Type” may identify whether an item is a drug or another type of supply. “RFID Tag #” not only identifies the number for an RFID tag associated with a particular item, but if blank may indicate that a certain item does not have an RFID tag. “Location” may identify a variety of information such as any kit or tray containing the specific item, a crash cart or inventory bin containing the specific item, and/or the current physical location of the specific item.

One of ordinary skill in the art will recognize that in other embodiments different types of information may be selectively presented to the user without departing from the inventive concept. Configuration selections are unique to each user, based upon their preferences and needs, and saved by the system so that they do not need to be entered each time a user accesses the system unless the user desires to make a change. Configuration selections allow each user the ability to control what they see, and make the most efficient use of the system according to their roles and responsibilities.

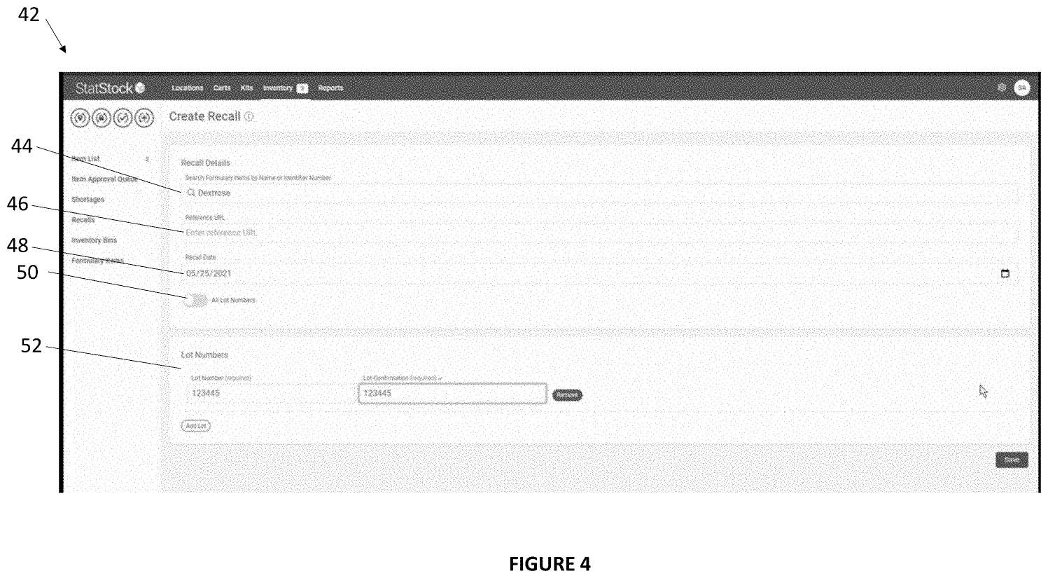

When a recall occurs for a drug or any other type of information, it is often crucial to identify all recalled items in inventory without delay so that they can be removed from circulation and any further use. It may also be important to identify recalled items even if they are not in inventory, in order to ensure that such recalled items are not accidentally added into inventory and used. As part of this, it is important to have a way for all users of the system to be informed about the recall. Referring to FIG. 4, an exemplary Create Recall page 42 allows a user to create a recall event in the system. First, a user may use a search field 44 to search the database of items to select which type of item is subject to recall. As an example, a user may search and select “Dextrose” if the recall is for a particular lot of Dextrose. The search may be done by drug name or by an identification number, such as a NDC (National Drug Code) number or other number that identifies a drug. A reference URL or other note may be entered into a reference field 46 to identify information about the recall. For example, if a drug manufacturer has posted information to their website about a drug recall, a user can enter that website URL onto the Create Recall page in order to provide other users with a source of information if they desire to know more about the recall. A date field 48 allows a user to enter in the recall date.

While many recalls pertain to particular lots of drugs or other items, in some circumstances a recall may pertain to all lots. A user can identify whether the recall pertains to all lots of a type of item or only specific lots. If all lots are impacted, the user can manipulate a button to identify this fact. If only a certain lot or lots, a user can identify the lot(s) by entering the lot numbers into lot fields 52. To ensure accuracy of information, the Create Recall page may require a user to enter each impacted lot number twice. Once all impacted lot numbers are entered into the system, a user can save the recall event. The system will then provide notifications as to any items in inventory that are subject to recall.

In exemplary embodiments a recall event may be created by scanning the bar code of a recalled item, or reading the RFID tag of a recalled item. Information stored in the system database associated with the scanned item may be populated onto the Create Recall page.

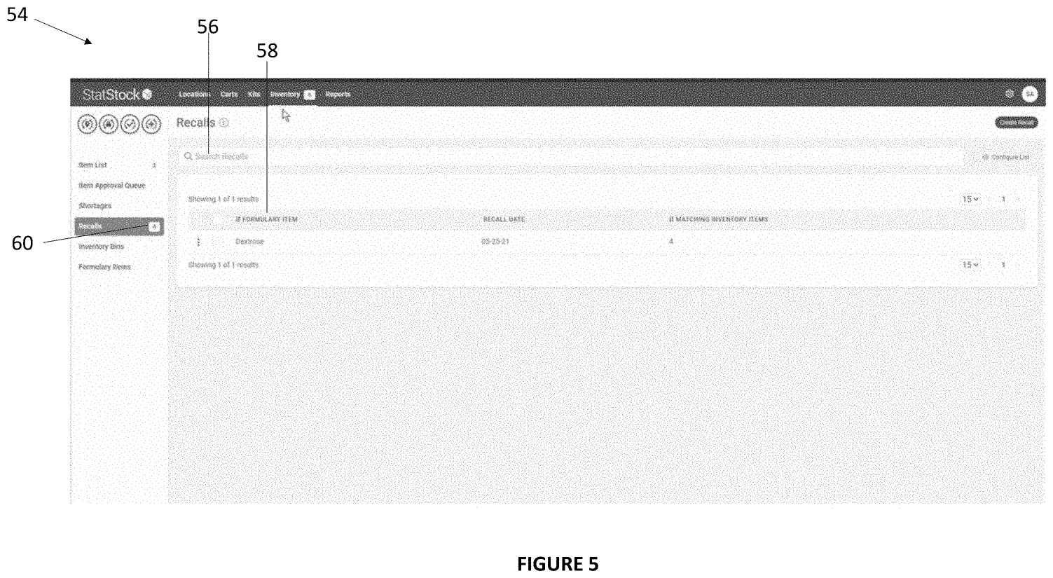

Referring to FIG. 5, an exemplary Recalls page 54 provides information about recall events. A search bar 56 allows a user to perform a search for any recalls of interest. Recall information for all recalls and/or searched recalls is provided in a recall list 58, which displays information that may include, but is not limited to, the type of item subject to the recall, the recall date, and the number of items in inventory that are subject to the recall. In the example shown in FIG. 5, a recall for Dextrose impacts four items in inventory. The number of recalls is also displayed as a notification 60 on the side bar 36.

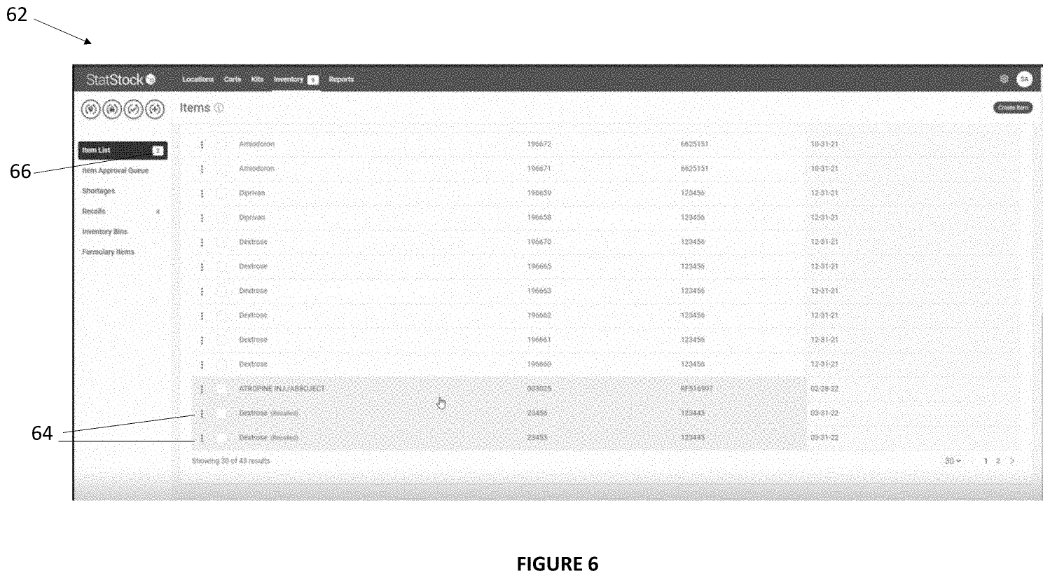

FIG. 6 is an exemplary Items page 62 showing notifications of recalled items after the creation of the exemplary recall event shown in FIG. 4. Items of Dextrose 64 are highlighted in red, and identified as “Recalled.” A notification 66 on the side bar 63 identifies that there are two items in inventory that are subject to recall.

Once a recall event has been entered, the system may prevent a user from adding any items subject to the recall into inventory.

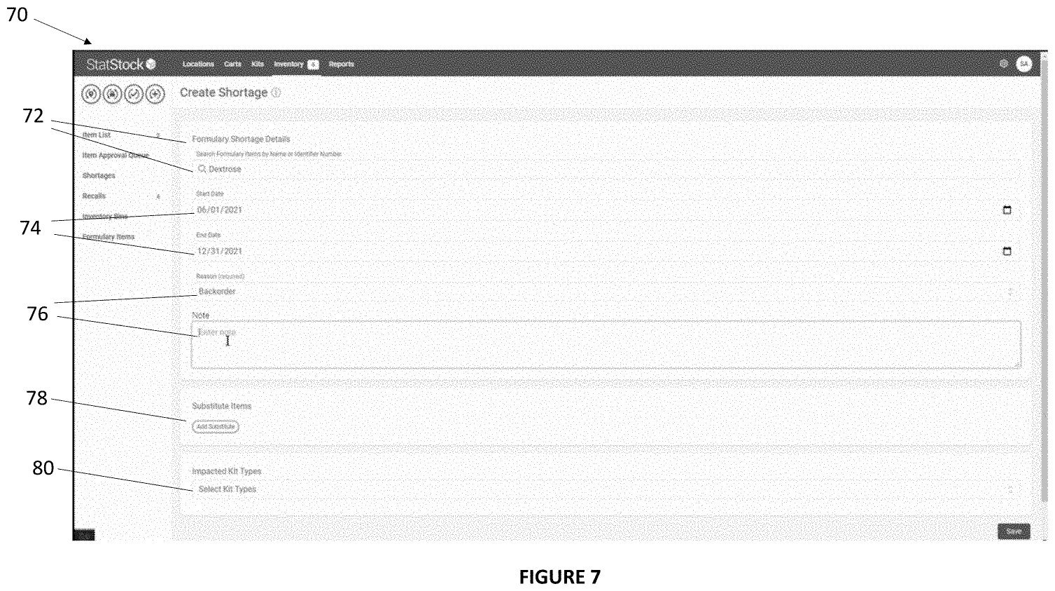

In the instance of a drug shortage, a shortage event may be created in the system by a user. Referring to FIG. 7, an exemplary Create Shortage page 70 is shown. A search field 72 may be used to identify the item subject to shortage by name or identification number. Date fields 74 may allow a user to enter start date and end date information for the shortage. An end date may not need to be entered in circumstances where the end of a shortage is unknown or cannot otherwise be anticipated at the time of creating the shortage event. Additional information fields 76 may allow a user to enter the reason for the shortage (such as a backorder), and any other notes about the shortage that might need to be communicated to others or otherwise recorded.

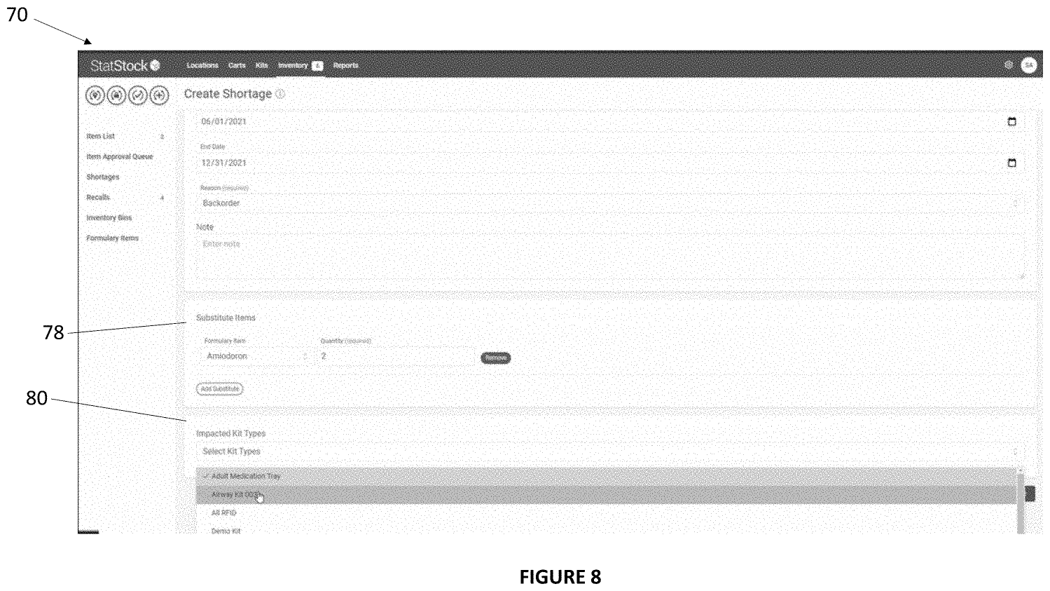

It is often necessary that in times of a drug or other item shortage, a substitute item is identified and permitted for use during the pendency of the shortage. Referring to FIGS. 7 and 8, a user may identify substitute items as part of creating a shortage event. In this manner, substitutions may be universally recognized across the system, without regard to any particular kit, tray, crash cart, inventory bin, etc. That is, the system allows for a substitution to be input as a single event, and subsequently apply across the entire inventory of items regardless of their location or association with other items if desired. A Substitute Items window 78 allows the user to identify the substitute by both name and quantity. While some items may be replaced with a substitute in a 1:1 ratio, in some circumstances the ratio may be different due to drug strength, the size of the item, etc. Merely as an example, a substitute for an individual item of Dextrose may be 2 items of Amiodarone, creating a 1:2 substitution ratio for the substitution event. Allowing an item to be substituted with multiples of another item provides greater flexibility. Referring to FIG. 8, once a substitute is identified (or substitutes are identified), the user can identify the instances in which the substitution is to be applied. For example, the system may provide a “Impacted Kit Types” field 80 that allows the user to select which type of kits will be impacted by the substitute. The user may select one, multiple, or all kits as desired. There may be options to simply apply the substitute to all kits. In other exemplary embodiments the system may allow a user to apply the substitution to any segment of inventory based on any particular common characteristic (for example, all kits intended for adult use, or all crash carts in the ER), or to apply the substitution to all inventory. One benefit of creating a shortage event and related substitution event is that it gives flexibility for either item (the item subject to shortage or the substitute item) to be used. Using the example of FIGS. 7 and 8 where 2 Amiodarone may be substituted for 1 Dextrose, some kits may contain Dextrose during the pendency of the shortage and others may contain Amiodarone. The system allows either to be used which reflects the situation in which a drug shortage does not mean that there is no subject drug available, but only that there is lower stock available and it may not be possible to continue using the subject drug in all kits, trays, inventory bins, etc. Allowing flexibility also means that as the shortage is lifted and the subject drug returns to pre-shortage availability, there is less disruption to the work flow.

As previously recognized, while not all items tracked by the system are associated with a kit, tray, etc., they are nevertheless part of tracked inventory. Users may inventory and track items that are unassociated with any kit, tray etc., and are instead associated with an inventory bin containing a number of items, which may all be the same type of item. Items in inventory bins may be input into the system in anticipation of eventual placement into a kit or tray, or it may be items that will never be placed into a kit or tray, such as larger items or general supplies. To the extent RFID-tagged items are included in inventory, the inventory bins allow for a more efficient process of tagging items. That is, the frequency at which items need to be tagged can be decreased if more items are tagged at a particular time, and certain tagged items placed in an inventory bin for later inclusion in a kit, tray, etc.

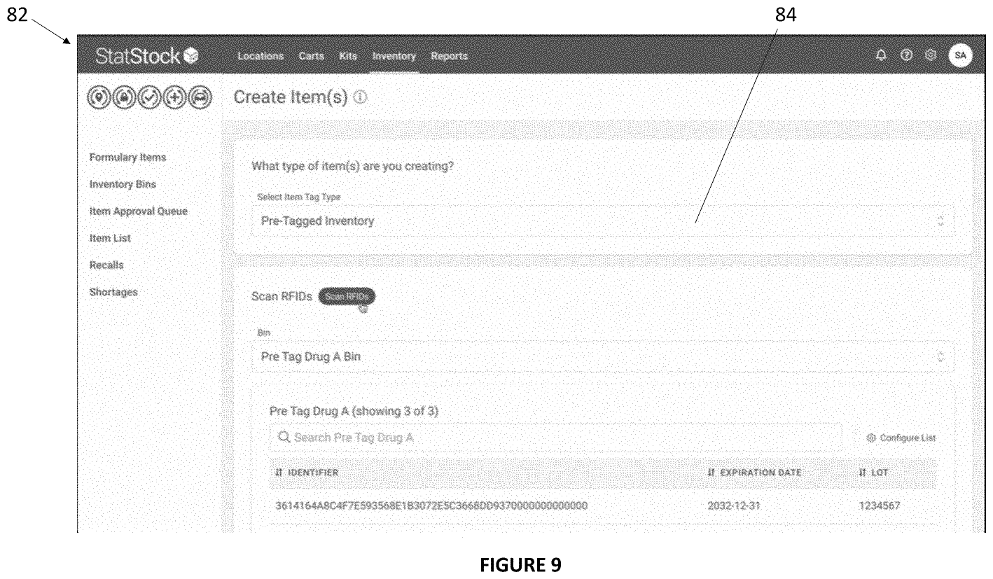

The system may permit RFID tagged items to be added into inventory in a variety of ways. Referring to FIG. 9, a Create Item(s) page 82 is shown. This page may be accessed by selecting “Add Inventory” 16 on the Dashboard page 10. On the Create Item page 82, the user may be prompted to identify the type of item that they are creating from a drop-down menu 84, namely, whether the inventory is pre-tagged or user-tagged. Pre-tagged items include those items where the manufacturer has already included an RFID tag on or in the drug label or other packaging. The RFID tag on pre-tagged items typically contains a variety of information about the item, including the drug name, the lot number, and the expiration date. User-tagged items include those that arrive from the manufacturer or distributor with no RFID tags and have to have an RFID tag manually applied to each item prior to being added into inventory.

One or more user-tagged items may be added into inventory by a user manually entering or scanning any barcodes on the item(s) to input information such as the drug name, national drug code, lot number, and expiration date. Once the tagged items are scanned by an RFID reader the items are saved in the system. The user may also associate the items with a particular bin. Pharmacist approval may be required to complete input of the items into the system.

A user may input pre-tagged items by selecting the “pre-tagged inventory” option on the Create Items page 84, and scanning the pre-tagged items. As shown in FIG. 9, the system may display information regarding each of pre-tagged items that has been scanned, including the identifier, the expiration date, and lot number. A user may configure the list to display a variety of additional information as desired, such as name, NDC code, etc. The user may associate the pre-tagged items with a bin. The user may save all of the pre-tagged items into the system. Pharmacist approval may be required to complete input of the items into the system.

The system may also permit items to be used that have not previously been added into inventory. That is, any time a pre-tagged drug is first scanned by the system, the pre-tagged drug may be automatically added into the system without requiring the user to create the item or otherwise approve the item for use. This provides a more efficient process that can save significant time for users who work with pre-tagged drugs. Automatic inputting of pre-tagged drugs may be permitted only when the RFID tags contain pre-determined necessary information (e.g., drug name, lot number, expiration date). A pre-tagged drug that lacks the predetermined necessary information may be flagged by the system and only input after a user has manually addressed any deficiencies.

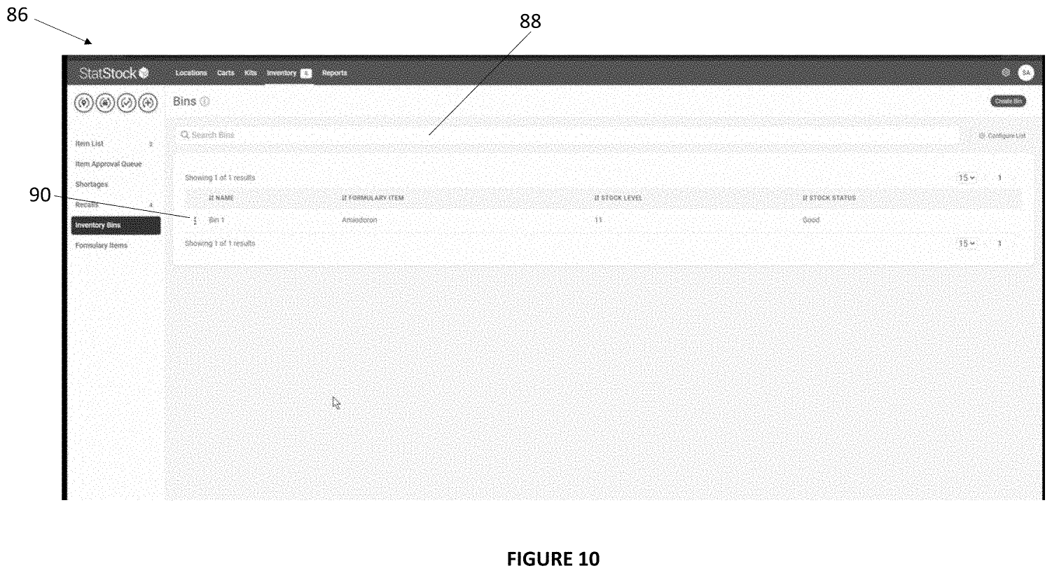

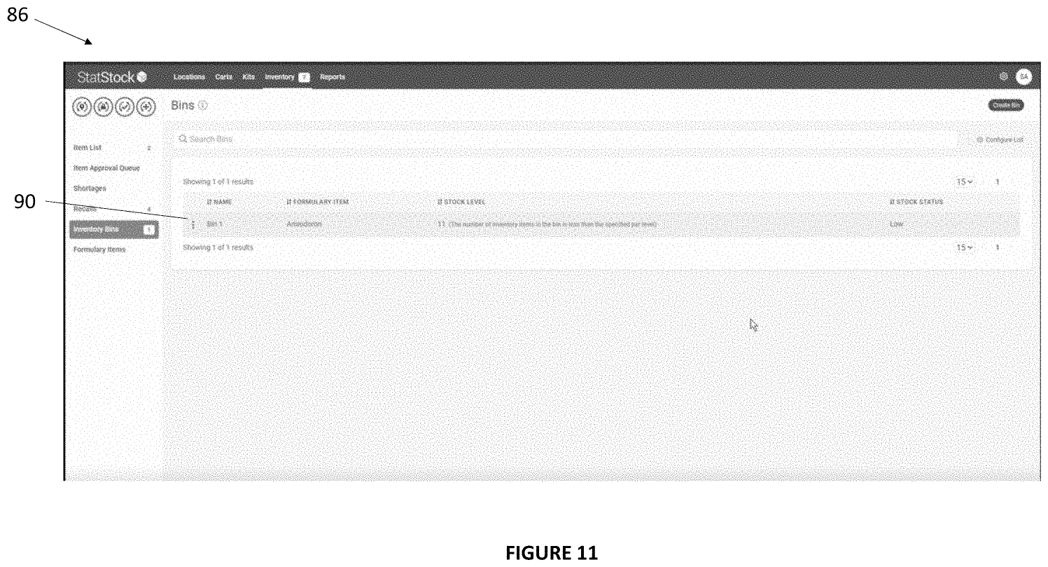

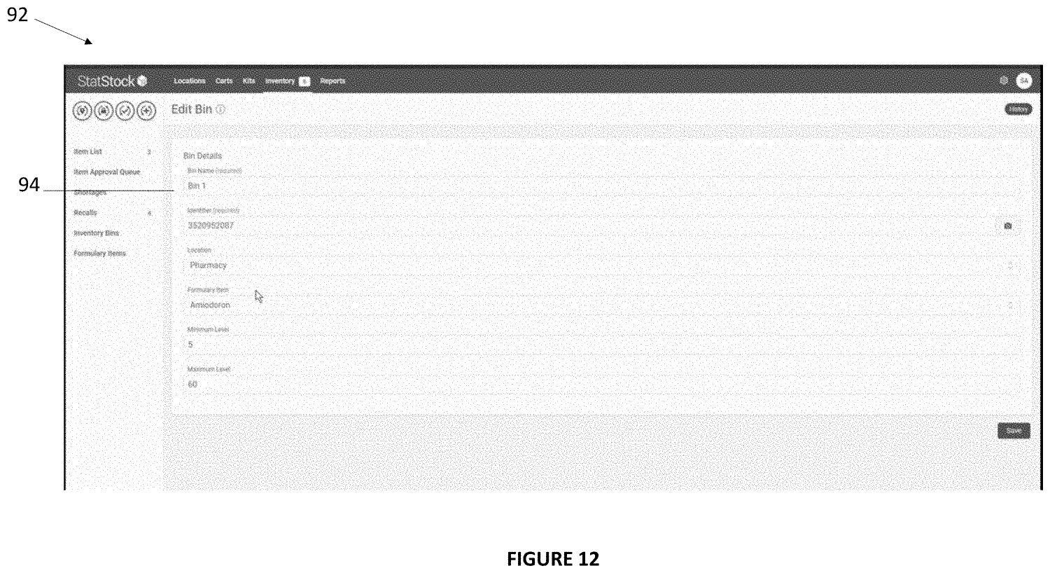

Referring to FIG. 10, an exemplary Bins page 86 is shown. This page 86 allows a user to search existing bins through a search field 88. Any results are displayed in a list 90 below. As shown in the exemplary Bins page 86, information such as the bin name, type of item (e.g., Amiodarone), stock level, and stock status may be displayed. The stock level identifies how many of the particular item are associated with the bin. The stock status identifies if the stock level comports with a par level that has been previously identified for the bin. For example, if a par level of 10 has been identified for a particular bin, and there are 11 items in inventory that are associated with the bin, then the stock status may be set as “good.” If, however, there were only 7 items in inventory for a bin that has a set par level of 10, then the stock status may be identified as “Low” or otherwise indicate a problem. Referring to FIG. 11, the stock status of Amiodarone has been identified as “Low” by the system after the par level is increased to more than 11 items. The value in having a par level for a bin is that there is always a desired amount of a particular item available. Setting different par values for different items allows users to configure the system to reflect the different rates of usage that are experienced across different types of items. In an exemplary embodiment, a user can set both a minimum and a maximum level for an item. Referring to FIG. 12, an Edit Bin page 92 according to an exemplary embodiment is shown. The Edit Bin page 92 allows a user to change the characteristics of a particular bin. Different Bin Detail fields 94 allow a user to edit information such as, but not limited to, the bin name, or a numeric identifier for the bin. A numeric identifier may be autogenerated (such as from the use of a barcode scanner) or manually entered. Additional information may be identified such as the bin's location, the type of item that is in the bin, and the minimum and maximum level of items for the bin. The minimum and maximum level of items in the bin may be entered to not only ensure that there is sufficient supply of a particular item already in inventory, but to prevent an unnecessarily high level of supply that may result in items expiring before use. Again, each item bin can be individually configured to reflect the expected amount of usage of a particular item. When a user views the bin list, different colors may be used to reflect that a particular bin has anything but a “good” stock status. For example, bins with a low stock status may be visually represented in red or another bright color to visually attract attention. A user may configure the Bins page to display the information that is most important and/or relevant to them. For example, they may choose to not display the min and max values, or they may configure the page to display them.

They system may also generate bin reports upon request or according to a predetermined schedule to identify the status of all inventory bins, including which bins may have a low or high stock status. The system may also generate notifications that are sent to one or more users in the event that the stock status falls into a “low” range. In certain exemplary embodiments, a “low” stock status may initiate the generation of a supply notice, to initiate the purchase of additional stock of the particular item.

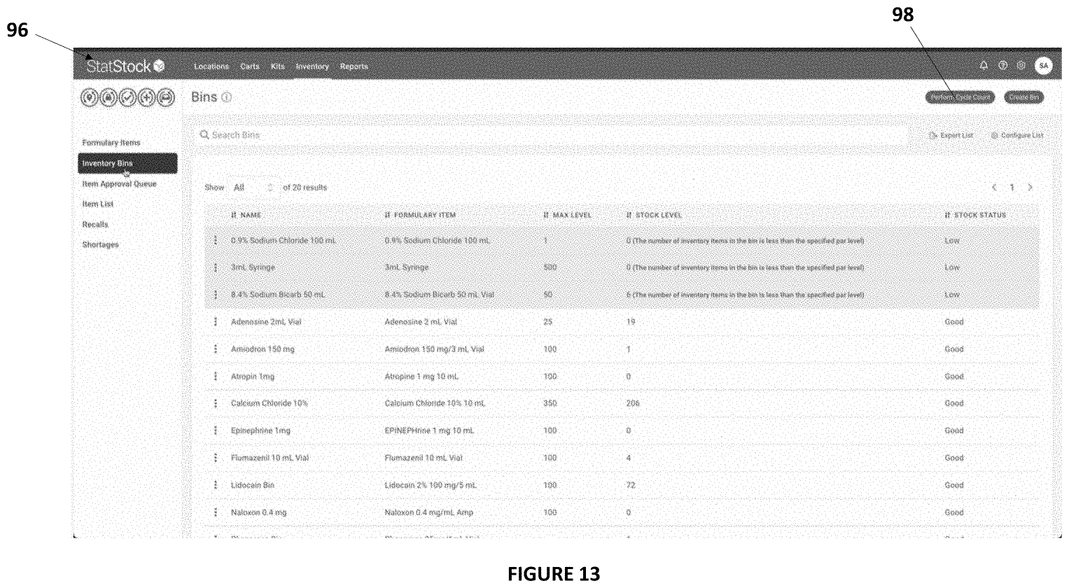

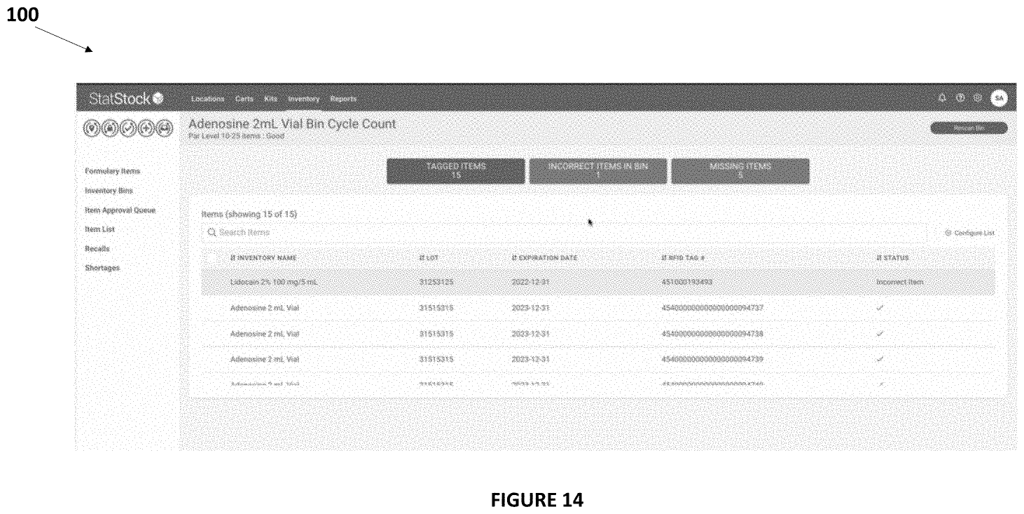

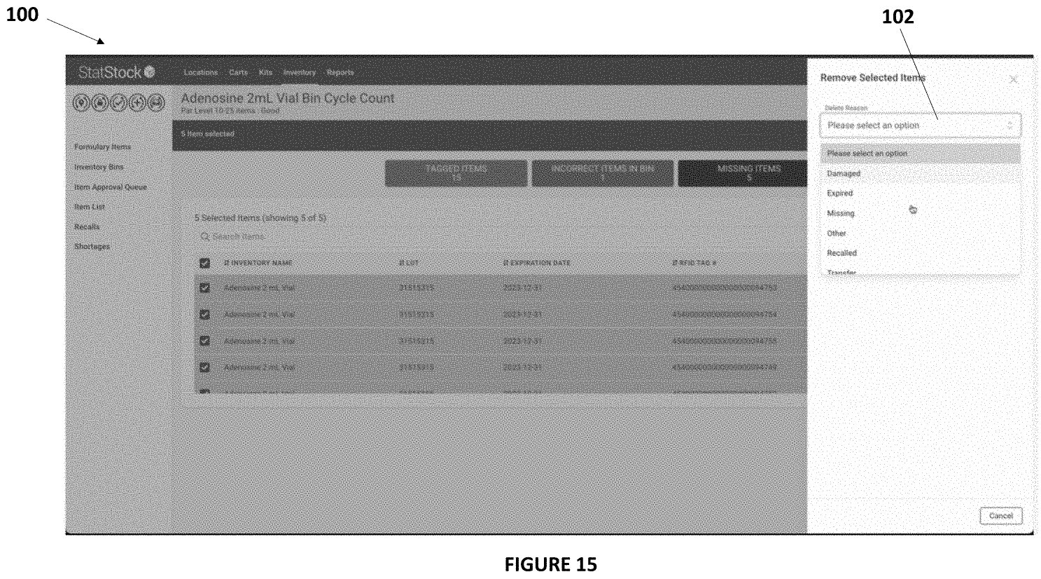

In an exemplary embodiment, the system may enable a user to perform a bin cycle count to assist in determining whether items from a particular bin had actually been placed into an intended kit, or whether the items had been displaced or diverted. Referring to FIG. 13, an exemplary Bins page 96 includes a button 98 for performing a cycle count on a selected bin. An exemplary bin cycle count results screen 100 is depicted in FIG. 14. The bin cycle results may show the number of tagged items in the bin, the number of incorrect items located in the bin (if any), and the number of items that are missing from the bin (if any). This feature allows a user to place an incorrect item into the correct bin, or investigate whether they can find any missing items. A user can also select items associated with the bin, such as “missing” items, that a user wants to officially remove from the bin. Referring to FIG. 15, a popup screen 102 invites a user to identify the reasons why one or more selected items are desired to be removed from the bin. Reasons may include, but are not limited to, an item being damaged, expired, missing, recalled, transferred, or other. Once a user identifies reasons for removal and confirms removal, the system may perform a subsequent scan in order to provide an updated count of all bin items.

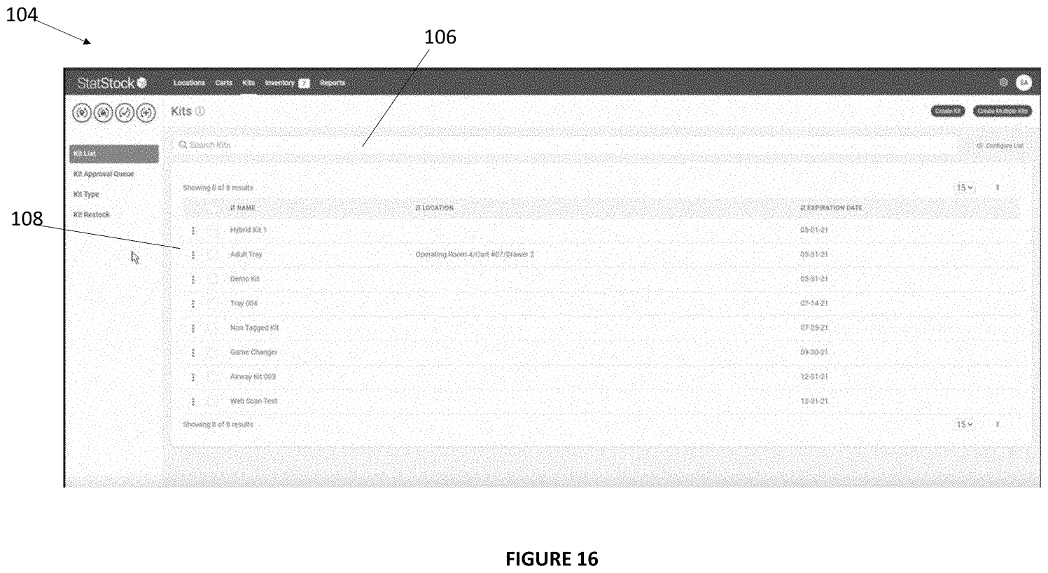

Kits may be tracked by the system to aid users in identifying their location, and whether any items contained in the kits are expired/soon expiring. Referring to FIG. 16, a Kits page 104 according to an exemplary embodiment is shown. A search field 106 at the top of the page allows a user to search for one or more kits. Kits may be searched by any variety of information that the system stores about the kit, including but not limited to, the kit name, kit type, location, or the RFID tag number of any RFID tag associated with the kit. A list of kit results list 108 is displayed according to the list configurations set by a user. For example, and as shown in FIG. 12, a user may choose to view the kit name, kit location, and next expiration date for each kit in the results list 108. The expiration date displayed is the earliest expiration date of any of the items in the kits that have had their expiration date entered into the system.

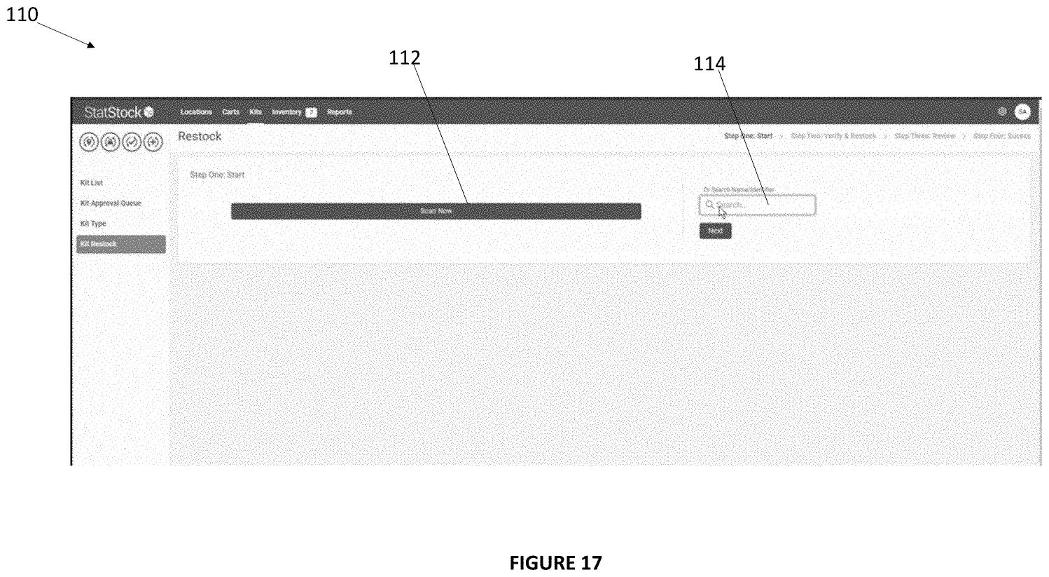

The system supports the restocking of Kits. Restocking of a Kit involves a comparison of items present in the kit against a template of what items should be in the kit. Different types of kits have different templates. In order to properly restock a kit, it is important for the system to identify the correct template for comparison. Referring to FIG. 17, a Restock page 110 according to an exemplary embodiment is shown. An initial screen gives the user two alternate ways to initiate the restocking process. The user can select a “Scan Now” button 112, which will initiate a scan of an RFID tag or barcode associated with the kit and will be used by the system to identify which type of kit it is, and accordingly, which template to use as a comparison. Or, alternatively, a user can manually enter a name or other identifier (such as a unique number) into a search field 114 associated with the kit to identify the kit and the proper template.

Restocking of kits containing RFID tagged items may be performed by restocking one or more missing items with pre-tagged items that have not been previously input into the system. A user may place the one or more pre-tagged items into the kit, and when the restocked kit is scanned the pre-tagged items may be automatically input into the system and used by the system to confirm whether the kit is complete and/or whether any changes need to be made to account for expired items, recalled items, etc.

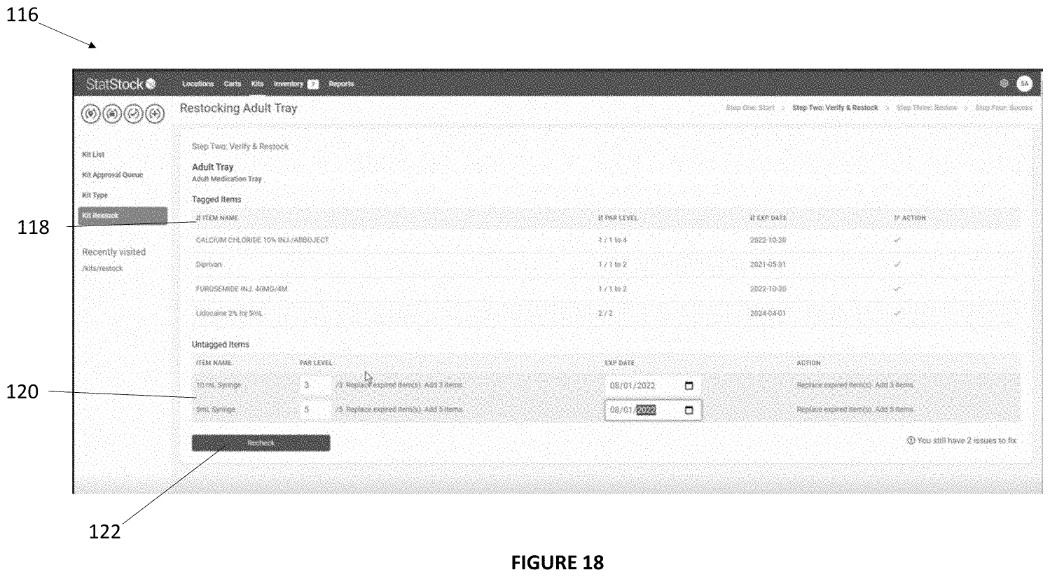

Once the system has identified the kit, the system can provide the user with information including, but not limited to, which items the system reports to be present in the kit, which items are missing, which items are subject to recall, and which items are expiring or soon to be expiring. The system's report on the status of particular items in the kit is based on data saved during earlier restocking(s) of the same kit, and any information received by the system since to indicate that items have been removed from the kit. If the system is associated with an RFID reading enclosure, or other type of RFID reader device, the system's report on the status of particular items may also be based on a present RFID reading. Referring to FIG. 18, an exemplary embodiment of a Restocking Adult Tray page 116 is shown. The system has recognized the kit as an adult tray, and in this example a user has placed the kit into an RFID enclosure device in order to scan the contents. The system subsequently identifies for the user each tagged item present in the kit, providing a list 118 containing tagged item names, par level, expiration date, and whether any action needs to be taken with respect to each tagged item. The system also provides a list 120 of any untagged items that it believes to be in the kit. In the example of FIG. 18, the untagged items are 10 ml syringes and 5 ml syringes. The system provides instructions for a user on the restocking of items needed, including how many to restock. For the untagged syringes, the system also prompts the user to enter any expiration date. When included multiple untagged items, the system may allow a user to enter each item individually and enter its corresponding expiration date, or choose to enter items of the same type as a group, and report only the soonest expiration date of all items in the group. Once all items are restocked, including the removal of any expired items or other items for which removal is requested, a user can select the Restock button 122 to identify that the kit is now completely restocked and any action items have been addressed.

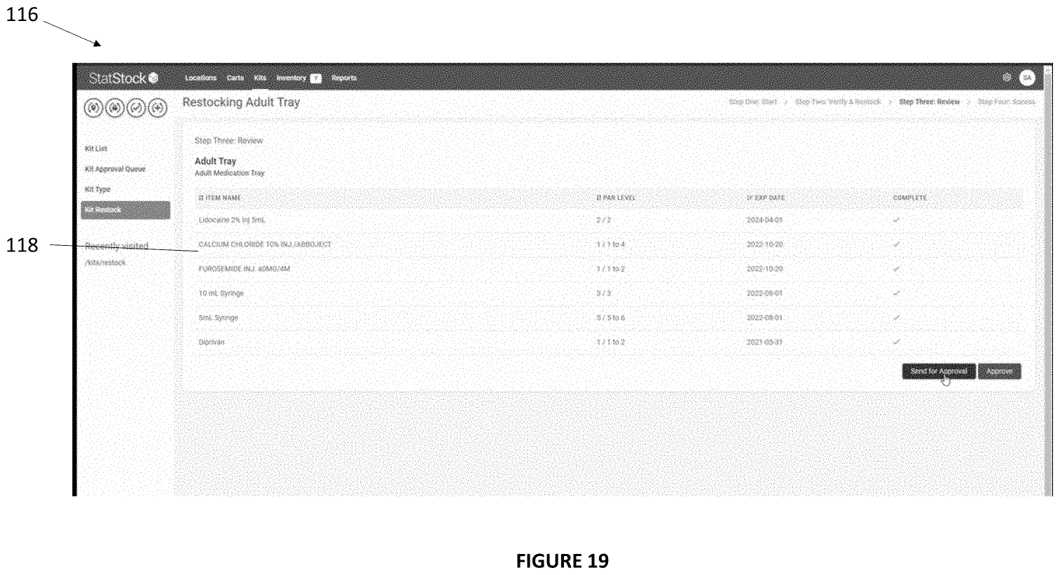

Once all items are restocked and verified, the system gives the user the ability to review all contents before the kit is officially approved for use. Referring to FIG. 19, the Restocking Adult Tray page 116 is shown after the kit has been restocked and verified. A review list 118 is provided of all items, both tagged and untagged, for the user to make one last confirmation. The user may then choose to approve the kit themselves, or send the kit to another user for subsequent approval. Depending on the authorizations given to a particular user, a user may or may not have the ability to approve a kit themselves. For example, for kits including drugs, it may be that only a pharmacist may perform approval of the kits before use. In such a circumstance there may be multiple users who perform kit restocking, but a smaller number of persons who can do approvals. Approvals that cannot be done by the current user may be placed into an approval queue.

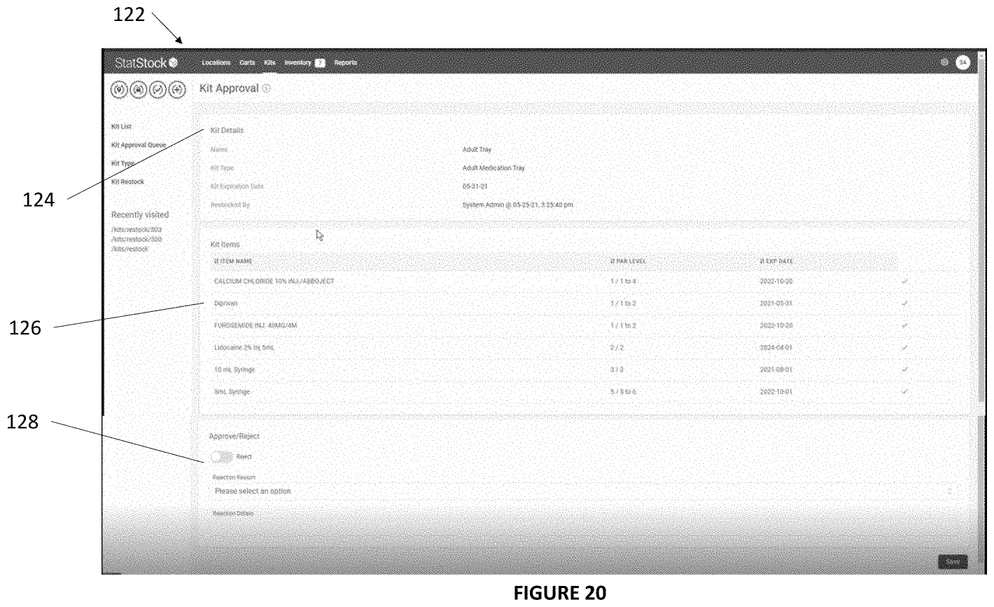

Referring to FIG. 20, an example Kit Approval page 122 according to an exemplary embodiment is shown. This page is associated with a particular kit in the approval queue. At the top of the page 122 is a Kit Details section 124 that provides basic information about the kit, including the kit name, kit type, kit expiration date (which may be the earliest expiration date of any item in the kit), which user performed the restocking of the kit, and the date/time upon which the restocking occurred. A kit items section 126 lists out the different items contained in the kit. Any notifications from the system about expirations, recalls, etc. pertaining to any of the items would be displayed in this section. The lack of any notifications for a particular item may also be represented by the system to give the user confirmation that no issues have been detected. At the bottom of the page 122 is an approval section 128 that allows the user to approve or reject the kit for use. If a kit is rejected, the user may identify a reason and/or provide further details to provide a record for the basis of rejection. In an exemplary embodiment, approval of a kit may require the user to re-enter their credentials to confirm they have authority to perform approvals. Once a kit is approved, its status as approved is saved in the system. A user may print off a report showing kit information, including the items in the kit that may accompany the kit. This may be done in situations including, but not limited to, when a kit is in the approval queue.

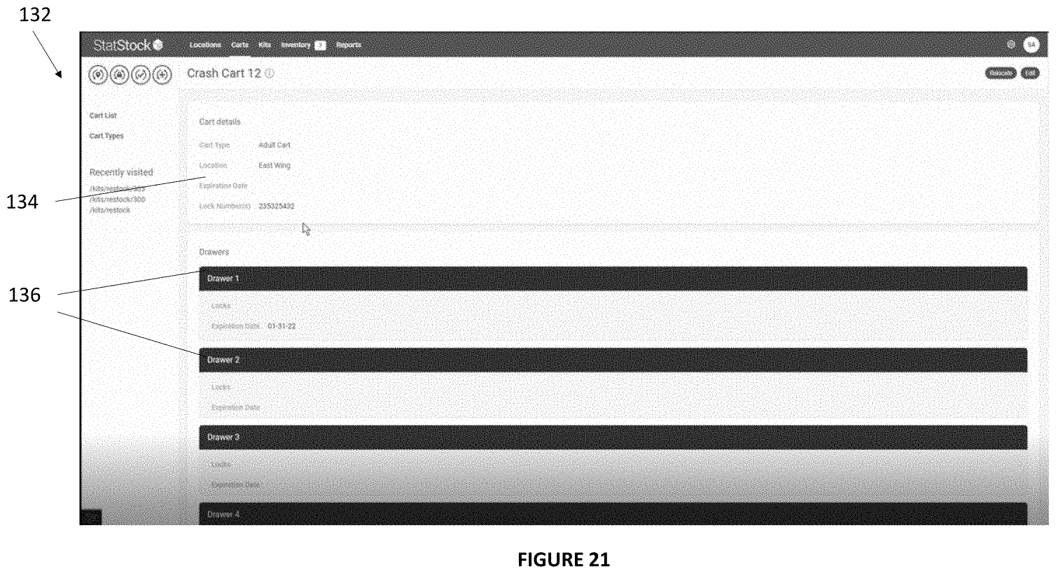

The system may provide users with a real-time inventory of what kits and/or individual items are in a particular crash cart, and even which drawer of a crash cart, and where the crash cart is located. This information may assist users in locating items that need to be removed due to recall or expiration. The system also allows users to designate crash carts and/or drawers of crash carts for specific types of kits and other items, to promote consistency and reliability, especially in urgent scenarios where a particular kit or individual item needs to be located without delay. For example, if a crash cart is designated as an adult crash cart in the system, the system may reject any attempt to designate a pediatric kit as located in the adult crash cart. Referring to FIG. 21, a Crash Cart page 132 according to an exemplary embodiment is shown. The Crash Cart page 132 displays information regarding the overall cart and each drawer in the cart. A cart details section 134 provides general information about the cart, including cart type, the location of the cart, and the earliest expiration date in the entire cart, and the Lock Number associated with any lock. A variety of tamper-evidence locks may be used with a cart, and each lock may have a unique number that can be entered into the system to comply with security protocols and otherwise create a security log for a cart.

A drawer detail section 136 provides information about each individual drawer contained in the cart, including any locks associated with each individual drawer, and the earliest expiration date of items located in the drawer. Different drawers may have different lock numbers and likely different expiration dates based on their individual contents.

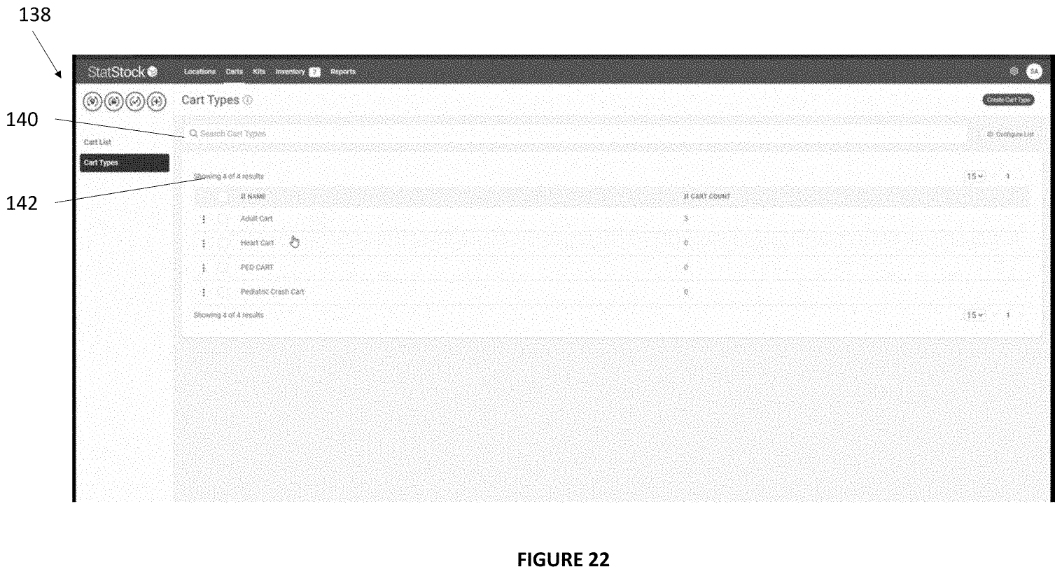

The system allows users to perform searches of carts based on cart type. Referring to FIG. 22, a Cart Types page 138 according to an exemplary embodiment is shown. A search bar 140 allows a user to search different carts based on their type. For example, a user may search for “adult” or “pediatric” or “heart” to identify carts of those types. A list of carts 142 allows a user to view how many carts of each type are recognized by the system. In the example of FIG. 22, three adult carts are in the system. If a cart of a particular type is needed in a certain location, a user can look up all carts of that same type to determine whether there is a cart that can be relocated.

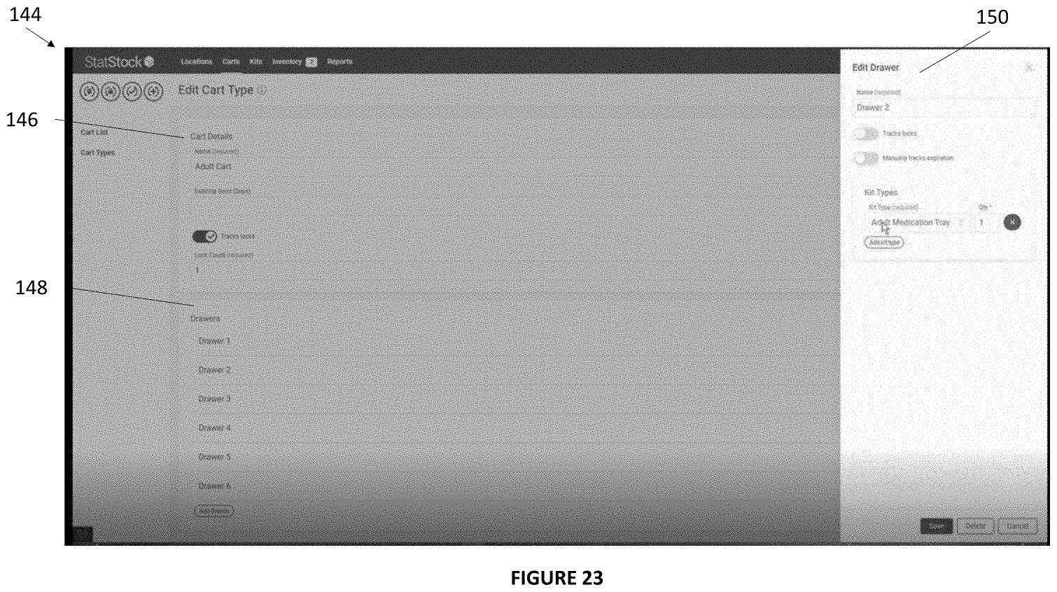

Referring to FIG. 23, an Edit Cart Type page 144 according to an exemplary embodiment is shown. This page allows a user to edit the properties of a cart. A Cart Details section 146 allows the user to edit information such as the name of the cart, and whether expiring items inside the cart will be tracked at the cart level. The user can also select whether the cart will track locks. If the cart does track locks, the user must identify what the lock count on the cart will be. A drawer section 148 of the page allows a user to select any drawer in the cart and edit the information about that drawer. An Edit Drawer window 150 allows the user to identify if locks will be tracked at the drawer level, if items in the drawer must be manually inspected for any expirations, and what kit types and number of kits may be placed into the drawer. Identifying manual inspection of expirations allows users greater flexibility in tracking expiration dates of drawer and drawer contents. For example, in addition to tracking the expiration date of a kit inside a drawer, users may also manually enter the expiration date of the entire drawer each time the drawer is accessed. This may be desirable when a drawer contains one or more items that are not part of a kit or tray.

In the example shown in FIG. 23, Drawer 2 of an Adult Cart may be designated to contain 1 Adult Medication Tray.

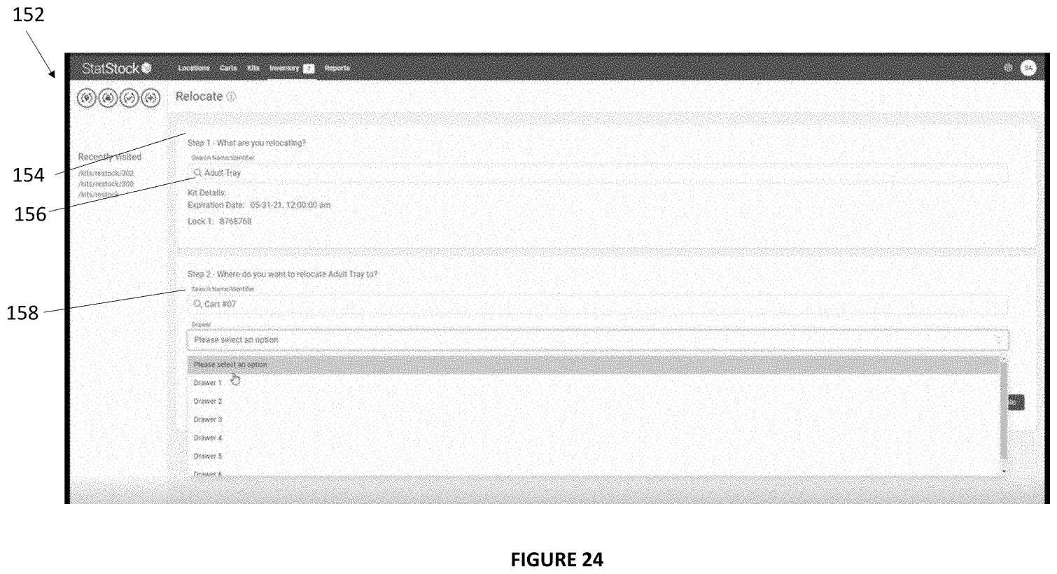

Whether a kit or tray has been recently approved after restocking, or is merely being moved from one location to another, the system allows a user to track relocation. Referring to FIG. 24, a Relocate page 152 according to an exemplary embodiment is shown. A first window 154 asks the user to identify what they are seeking to relocate. The user can search for the kit, tray, or other type of article to be relocated in a search field 156. The system may also allow the user to scan an RFID tag associated with the article, or scan a barcode, in order to do the identification. Details about the article may be presented to the user in order to help the user confirm that they have selected the right kit, tray, etc. A second window 158 will then ask the user to identify where they want to relocate the article to. A user may not only specify which cart they wish to relocate to, but a specific drawer in that cart. For example, a user may instruct the system to relocate a particular tray to Drawer 3 of Cart #7. Once the user has made their selection, the system may provide them with a confirmation window providing details on the requested relocation and asking the user to confirm that it is correct. The system may allow for relocation of virtually any kit, tray, cart, or even individual item. For example, the system may allow a user to relocate a specific cart from one location to another, such as from one operating room to another operating room. In doing so, the system allows users to track the location of all inventory, making it easier to locate any item when needed.

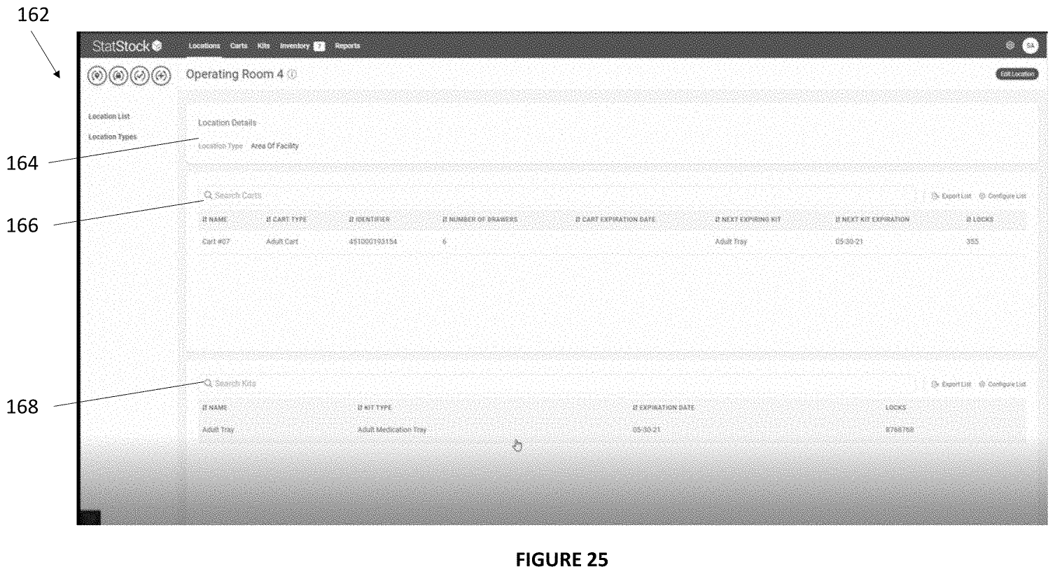

The system not only allows a user to relocate items from one location to another, but also allows users to view all assets located in any particular location. Referring to FIG. 25, an example of a location page 162 according to an exemplary embodiment is shown. This example is for the location identified as “Operating Room 4.” A Location Details section 164 provides the user with information about the location, such as the type of location. This section may provide any type of information to help identify or explain the location. In exemplary embodiments this section may include a link to a map, such as a facility map or campus map to help identify the location to a user. Search fields 166 and 168 allow the user to search for any carts, kits, or other types of assets that the system may identify as being at the location, which once searched may be listed out below. In some exemplary embodiments the system may allow a user to view history of the location to not only track what assets are presently at the location, but also view which assets were previously at the location. A user may select any cart, kit, or other asset to further view details about that specific asset, including specific items contained therein and related details.

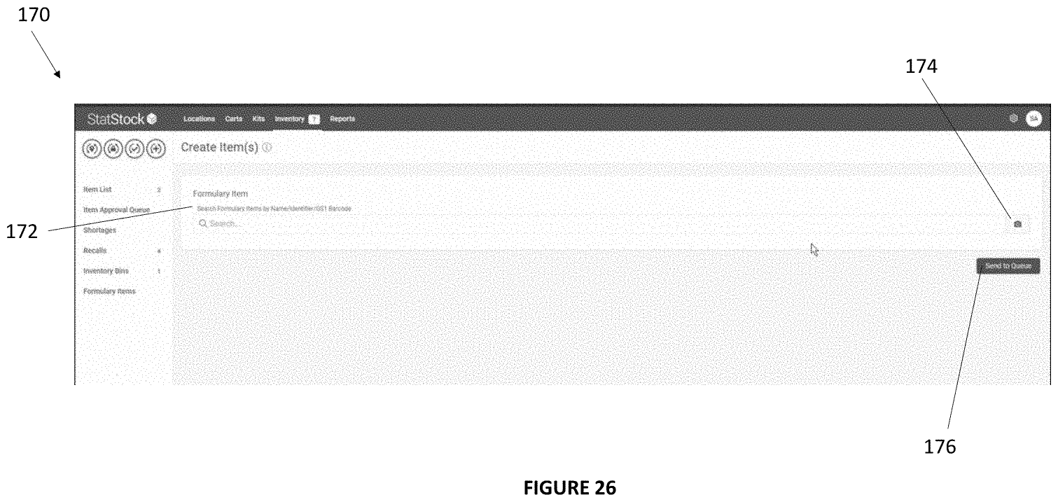

Referring to FIG. 26, a Create Items page 170 according to an exemplary embodiment is shown. The Create Items page may allow a user to identify a new item in a variety of different ways. Using a search field 172, a user can identify formulary items by their name, barcode number, or other identifier. The user can use a barcode scanner to scan a barcode as well to identify the item. If the user has an electronic device available with a camera, such as a mobile phone with a camera function, the user can click on a camera icon 174 and take a picture of the barcode on the item using their mobile phone or other device. As most persons carry mobile phones or other portable electronic devices with them, it can be highly convenient for users to take photographs of bar codes in order to identify items, providing for efficient mobile locating of items. Once an item has been identified, the user may select a button 176 to send the item on to an approval queue. The queue may allow another user, who may have different permissions and authority, to confirm the addition of the item into the system. For example, a pharmacist may review the queue and either approve or deny pending requests to add items into the system that have been initiated by other users.

In an exemplary embodiment, the system may be configured to only accept certain types of input from a barcode reader. A “barcode only” setting may be used to add an item, search for an item, relocate an asset such as kit or cart, or relock a kit or cart. A barcode only setting may instruct the application server and/or central server to analyze the speed at which data is input into the system, and reject any data that is not provided above a certain threshold of speed. This may effectively prohibit manual entry of certain types of information, and ensure that important data inputs (e.g., an identification number associated with a kit, cart, or item) can only be made by barcode. This feature may reduce the amount of human error caused during manual entry of information.

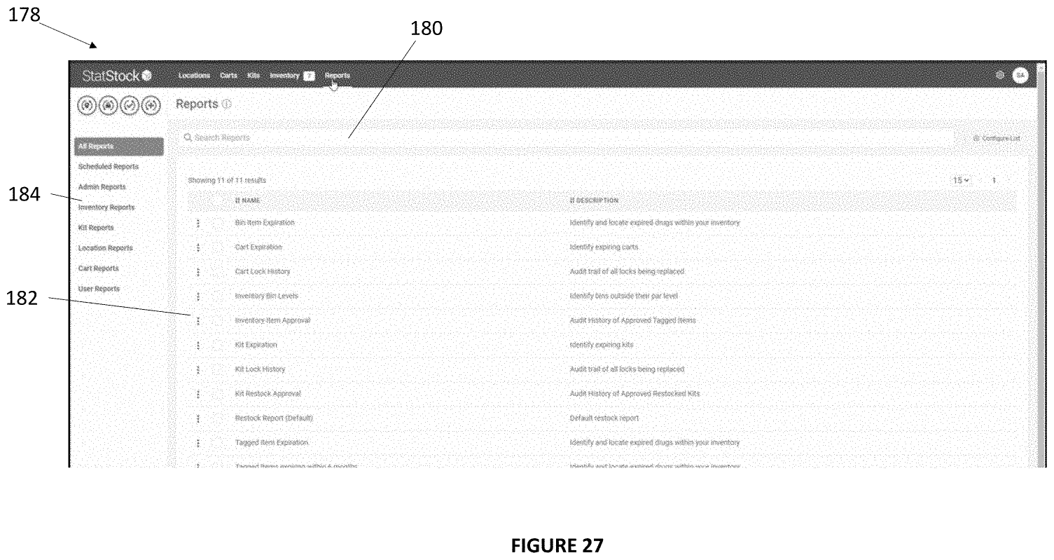

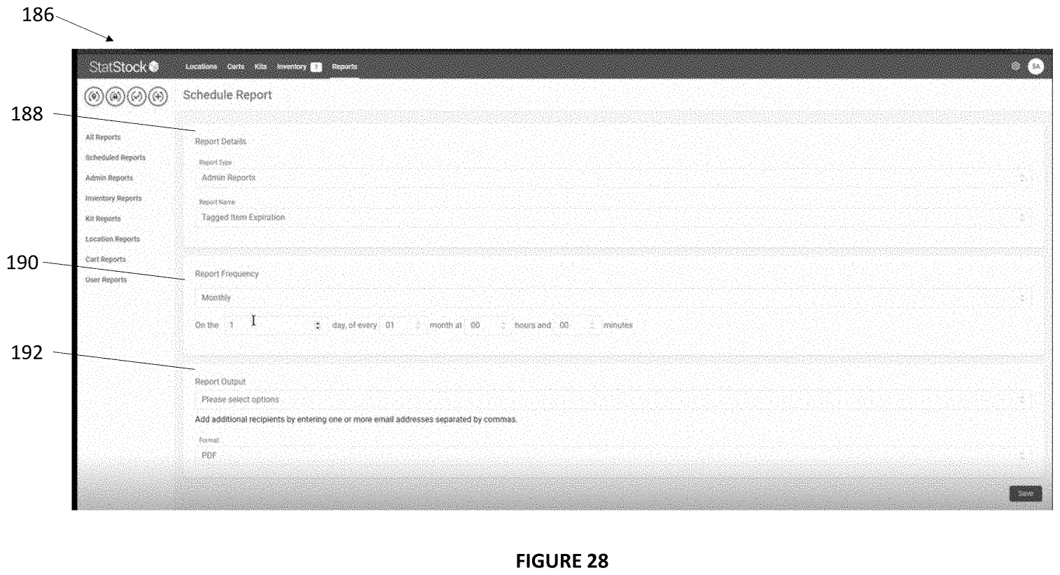

The system provides users with the ability to generate reports on virtually all information tracked by the system, configure and customize reports, and save reports for future use. Referring to FIG. 27, an exemplary Reports page 178 according to an exemplary embodiment is shown. A search bar 180 at the top of the page allows a user to search any existing reports, and a list of reports 182 are shown below. If a user desires to run an existing report they can select it from the list and instruct the system to run the report based on current information. A menu bar 184 lists different categories of reports to also aid a user in finding a report. Different reports that may be generated by the system include, but are not limited to, administrative reports, inventory reports, kit reports, location reports, cart reports, and user reports. The system may also generate scheduled reports, according to a schedule set by a user. Referring to FIG. 28, a Schedule Report page 186 according to an exemplary embodiment is shown. In a Report Details section 188 the user may identify the report type and the report name that they desire. The frequency at which the report is to be run can also be set by the user. In a Report Frequency section 190 a user may identify the basis such as daily, monthly, yearly, etc. The user may further identify more precise timing based on the general basis. For example, if the user desires a particular report to be generated on a monthly basis, they may further set the day of the month, and what time of day they wish the report to be run. How the report is to be communicated can also be set by the user. In a Report Output section 192, a user may identify which recipients should receive a report by providing their email addresses, and the user can also select the format of the report, such as .pdf, .html, xlxs, etc. In such an embodiment the report may be emailed to recipients as an attachment. In other embodiments a report may be communicated by text message with a link, or any other means that allows one or more recipients to view its contents. In other embodiments the system may notify users that reports are available for viewing through the Reports page.

Reports may be scheduled to show, among many other things, what items will soon be expiring. A user can select how far in advance of expiration dates the report will include.

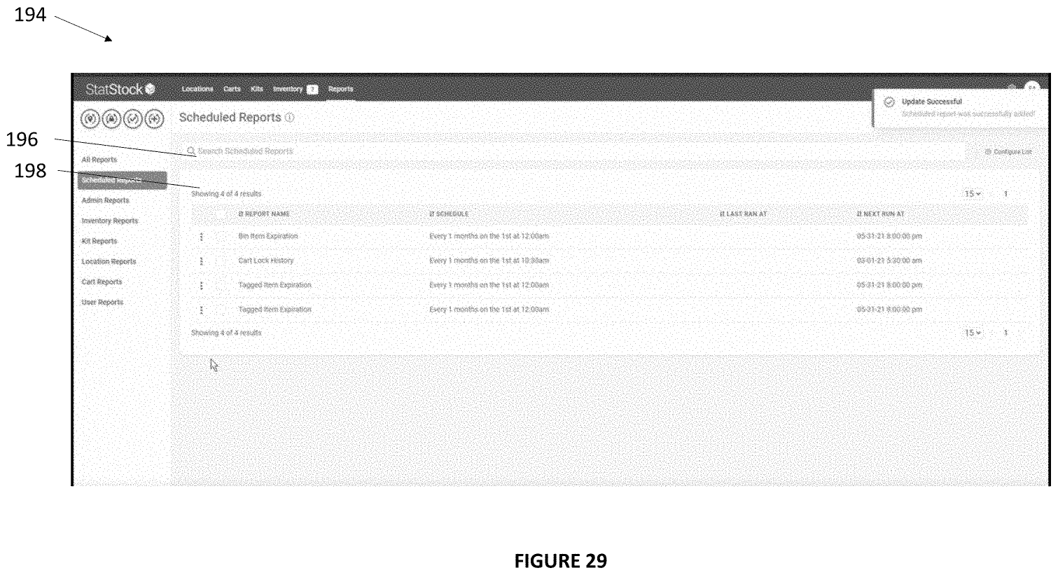

Referring to FIG. 29, a Scheduled Reports page 194 according to an exemplary embodiment is shown. A search bar 196 allows a user to search for any reports that are scheduled. A list of scheduled reports 198 may also be presented to a user. A user may select any scheduled report to view further details and make edits. If a user has recently created a new scheduled report the system may confirm that the new report has successfully been added.

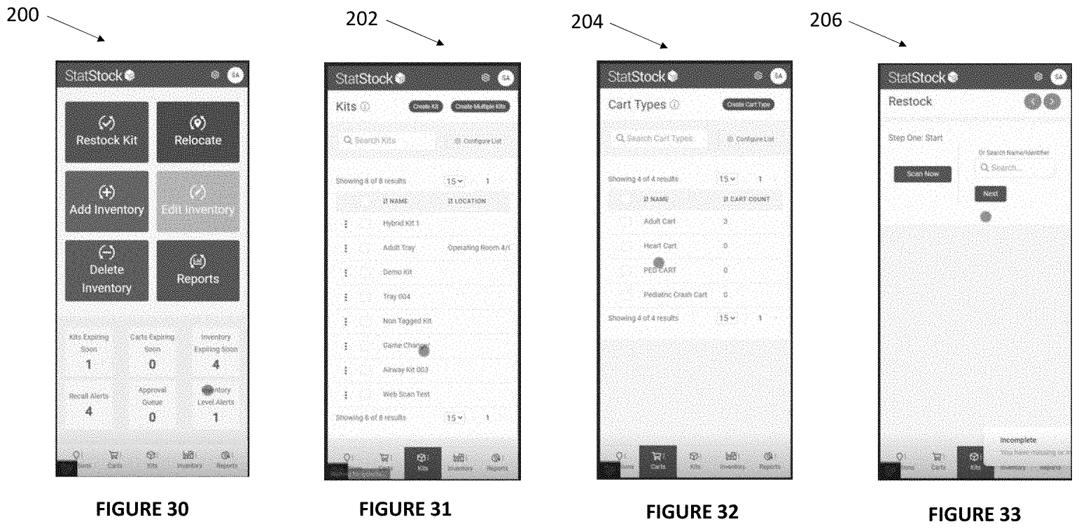

In an exemplary embodiment where the system is web-based and accessible from any internet-capable device, users may access the system not only from computers but from their mobile devices. FIGS. 30-33 are exemplary pages as may be shown on a mobile device. Through a mobile device a user may interact with any aspect of the system as they could on any other type of electronic computing device. FIG. 30 is an exemplary page of the dashboard 200 as may be seen on a mobile device; FIG. 31 is an exemplary Kits page 202; FIG. 32 is an exemplary Cart Types page 204, and FIG. 33 is an exemplary Restock page 206. In other exemplary embodiments the mobile pages may differ, but one of ordinary skill in the art will recognize that many changes as to format and design of the pages shown in all of FIGS. 1-33 may be made without departing from the inventive concepts described and depicted herein.

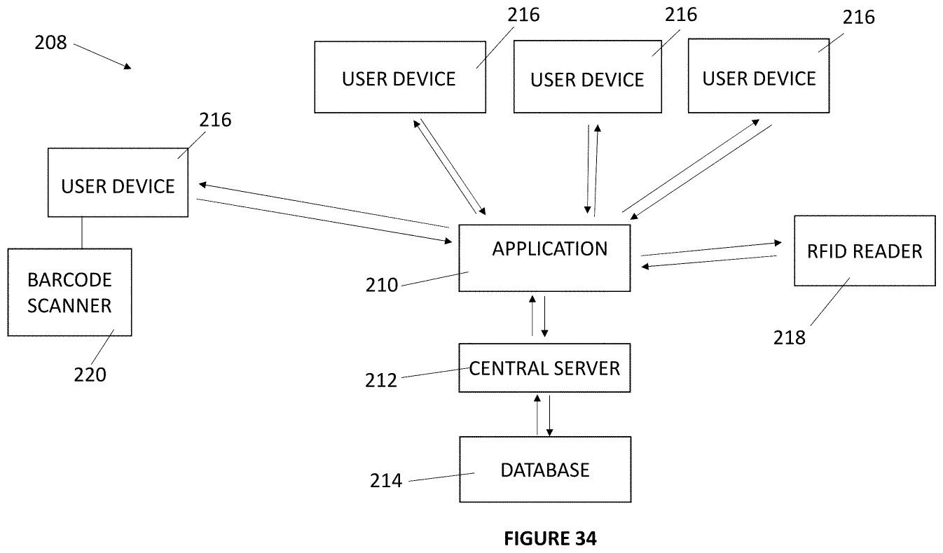

Referring to FIG. 34, a diagram of an exemplary system 208 for performing inventory management is shown. The system is comprised of an application 210 in communication with a central server 212 associated with a database 214. The application 210 may be a web application running on an application server or other server that may be executed within the web browsers of a plurality of user devices 216. The central server 212 performs business logic and communicates with the database 214 where information is stored. The application server communicates with any RFID readers 218 that are utilized in an embodiment of the system.

The application 210, central server 212, and database 214 may be any configuration of hardware and software that can perform the functions discussed in this disclosure and depicted in the figures. The application 210, central server 212 and associated database 214 may also comprise a variety of decentralized and/or distributed computing systems and databases. Nothing herein is intended to limit the application 210, central server 212 and associated database 214 to any one single embodiment of hardware and/or software.

The application 210 allows users to interact with the system, including viewing information, configuring the system, inputting information, instructing the system to perform certain operations and activities, and exporting information. A web application such as that shown in the exemplary embodiment of FIGS. 1-33 may be hosted by the application 210 and accessed by a variety of user devices 216 (e.g., smartphones, tablets, personal computers, desktop computer, laptops, etc.). Barcode scanners 220 operatively connected to a user device, such as a desktop computer, may provide for the input of barcode information into the system. Other types of user devices 216, such as smartphones, may also be used.

In an exemplary embodiment, the system can create and store user profiles, and provided different levels of access and administrative abilities to different users. For example, and as discussed herein, some users may lack authority to approve restocked kits or trays, or may lack authority to add items into the inventory system. Some users may have more permission than others to configure reports or edit information. One of ordinary skill in the art will recognize that the system could allow for a variety of user roles, permissions, and functionalities depending on the particular application. Usernames and passwords, and other forms of authentication and verification, may also be utilized to keep information secure.

Any embodiment of the present invention may include any of the optional or preferred features of the other embodiments of the present invention. The exemplary embodiments herein disclosed are not intended to be exhaustive or to unnecessarily limit the scope of the invention. The exemplary embodiments were chosen and described in order to explain the principles of the present invention so that others skilled in the art may practice the invention. Having shown and described exemplary embodiments of the present invention, those skilled in the art will realize that many variations and modifications may be made to the described invention. Many of those variations and modifications will provide the same result and fall within the spirit of the claimed invention. It is the intention, therefore, to limit the invention only as indicated by the scope of the claims.

1. An inventory management system, said system comprising:
a server in communication with a network, said server associated with a database, said server configured to:
receive first item information associated with a first item, said first item information comprising identification information, expiration information, and lot number information; said first item information received from one said at least one user device associated with a barcode reader;
receive second item information associated with a second item, said second item information comprising identification information, expiration information, and lot number information; said second item information received from one of said at least one user device associated with an RFID reader;
receive third item information associated with a third item, said third item information comprising identification information; said third item information received from one of said at least one user device capable of receiving manual entry of said identification information;
associate at least one of said first item, said second item, and said third item with a kit;
associate at least one of said first item, said second item, and said third item with a cart, and
provide a notification to a user.

2. The inventory management system of claim 1, wherein said server is further configured to:
associate at least one of said first item, said second item, and said third item with a bin, wherein said bin is defined by at least one identification characteristic; store identification information for every item associated with said bin in said database;
receive, from at least one of said at least one user device in association with an RFID reader, information about items present in said bin during a scan; and
compare said information about items present in said bin during a scan with identification information stored in said database;
provide a bin notification to a user.

3. The inventory management system of claim 2, wherein said bin notification is selected from the following group: notification of a missing item, notification of an item that does not have said at least one identification characteristic, and notification of number of recognized items.

4. The inventory management system of claim 2, wherein said server is further configured to:
receive a user device a par level instruction for said bin;
store said par level instruction in said database;
provide a par level notification to a user.

5. The inventory management system of claim 4, wherein said par level notification is selected from the following group: notification that the current number of items associated with said bin is less than the par level, notification that the current number of items associated with said bin is within a certain number of items of the par level.

6. The inventory management system of claim 1, wherein said server is further configured to:
associate at least one of said first item, said second item, and said third item with a kit;
associate at least one of said first item, said second item, and said third item with an inventory cart;
store a location associated with said kit in said database;
store a location associated with said inventory cart in said database;
provide, in response to an inquiry received from a user device, the location of said kit;
provide, in response to an inquiry received from a user device, the location of said inventory cart.

7. The inventory management system of claim 1, wherein said server is further configured to:
calculate the expiration status of at least one kit; and
cause a user device to display said expiration status of at least one kit.

8. The inventory management system of claim 5, wherein said server is configured to:
calculate the expiration status of at least one inventory cart; and
cause a user device to display said expiration status of said at least one inventory cart.

9. An inventory management system comprising:
a user device;
an RFID reader in communication with said user device;
a server in communication with said user device; said server associated with a database, and said server configured to:
receive kit instructions, said kit instructions comprising required items that must be present in a complete kit;
receive item information for a plurality of items detected in a scan performed by said RFID reader, wherein said item information for each of said plurality of items comprises unique identification information, expiration information, and lot information;
compare said received item information with said kit instructions to identify whether all required items were present during said scan;
calculate whether any of said required items were missing;
calculate whether any of said plurality of items had expired;
calculate whether any of said plurality of items had been recalled; and
initiate a notification to a user.

10. The inventory management system of claim 9 wherein said plurality of items includes at least one item not previously encountered by said server, and said server, in the event of confirming that all required items were present during said scan, saves said at least one item not previously encountered by said server in said database.
